(19) United States
(12) Patent Application Publication
(10) Pub. No.: US 2019/0175392 A1
(43) Pub. Date: Jun. 13, 2019

(54) AFTER-INSECT BITE DEVICE AND METHOD

(71) Applicant: ASCHES S.R.L., Grassobbio (IT)

(72) Inventors: Giacomo Cossolini, Bergamo (IT); Fabio Balzarini, Bergamo (IT); Silvia Carpino, Bergamo (IT); Umberto Trotta, Ponte San Pietro (IT); Roberto Balzarini, Stezzano (IT)

(73) Assignee: ASCHES S.R.L., Grassobbio (IT)

(21) Appl. No.: 16/302,280

(22) PCT Filed: May 24, 2017
(86) PCT No.: PCT/EP2017/062573

§ 371 Date: Nov. 16, 2018

(30) Foreign Application Priority Data
May 24, 2016 (IT) UA2016A004304

(51) Int. Cl. A61F 7/02; A61M 35/00; A61F 7/00; A61K 36/28; A61K 36/815; A61K 36/53

(52) U.S. Cl. CPC A61F 7/02; A61M 35/003; A61F 7/007; A61K 36/28; A61K 36/815; A61M 2205/05; A61F 2007/0071; A61M 2205/3368; A61M 2205/8206; A61F 2007/0284; A61K 36/53

(57) ABSTRACT

They are described an after-insect bite device (100) and method. The device (100) comprises a container body (10); an applicator head (30) extending from one end (14) of the container body (10); and a reservoir (50) housed within the container body (10) and adapted to contain a mixture (M) that is intended to be applied, through said applicator head (30), onto a part affected by the insect bite. The mixture (M) is a mixture (M) of completely vegetal substances and the device (100) further comprises heating means (40) housed within the container body (10) and which can be activated by switching on/off means (70) of said device (100) to heat said part affected by the insect bite, before applying said mixture (M).

### TECHNICAL FIELD OF THE INVENTION

The present invention generally relates to the technical field of the after-insect bite devices. In particular, the present invention relates to a device and a method for dispensing a mixture made of completely vegetal substances onto a part of the user's body which is affected by an insect bite, after heating the part affected by the insect bite.

### BACKGROUND OF THE INVENTION

Insects represent the five-sixths of the entire animal kingdom. Many of them have a nutritional ethology totally, or partially, or almost, apart from any interaction with humans, although, in some cases they benefit from human presence, their uses and habits, the ability of humans to accumulate dust (e.g., mites) or to produce large amounts of waste (e.g., cockroaches). Other insect species, known as biting insects, interact in an even more direct way with humans, reaching an often annoying and unexpected contact, which can cause discomfort situations and, in extreme cases, compromise the ability of carrying out the usual everyone's daily activities.

Biting insects are divided into two main categories: defense/attack biting insects, such as bees, bumblebees, wasps, and the like, which inject, through their sting, toxic and/or poisonous substances for defense and/or attack purposes; and hematophagous biting insects, such as mosquitoes, fleas, lice, sandflies and the like, which sting or bite to feed themselves on bitten subject's blood.

With particular reference to the hematophagous biting insects, such as a mosquito, the bite occurs at the skin level, and blood is usually taken from peripheral small blood vessels to be introduced into the insect organism through a buccal apparatus specialized in biting-suction of the nutrient. In addition to withdraw blood, the insect injects into the human body an anticoagulant and vasodilating protein present in its saliva, and which is the main cause of the discomfort caused by the bite, typically itching.

Typically, the sting or bite effects are felt subsequently to the sting or bite, and precisely when the substances external, and therefore extraneous, to the bitten organism are isolated and attacked by the lymphocytes, thus initiating a systemic immune reaction, localized in the bite area. This generates the wheal and the discomfort associated therewith, such as itching, diffuse discomfort and local temperature rise.

To date, the above mentioned issues connected with the insect bites are addressed and resolved chemically, that is, by using after-bite devices which employ mild basic chemical substances, typically ammonia, in solid or liquid form. Such chemical substances, when applied onto the wheal by rubbing the skin, aim to counterbalance the predominantly acidic nature of the saliva of hematophagous insects, resulting in the elimination of the aforementioned discomforts caused by the wheal. More specifically, the above chemical substances act by at least partially denaturing the anticoagulant and vasodilating protein present in the saliva of the biting insect, and which is injected into the body of the subject at the time of the bite. In particular, denaturation, for example of a protein, consists in loss of its three-dimensional structure sufficient to determine loss of function thereof.

However, the after-insect bite devices mentioned above have some drawbacks, essentially related to the chemical nature of the substances used therein.

First, these chemical substances, if not sufficiently diluted, result to be aggressive on the skin, and can therefore cause irritation when applied on particularly delicate skin, such as in the facial area, or on children's and infant' skin.

It has also been found that, due to poor skin permeability and low concentration of the chemical substances, the known after-bite devices mentioned above are hardly capable of resolving the compromised situation that arises as a result of the systemic trauma caused by the bite.

Furthermore, the limitations of after-bite devices employing chemical substances are increased by a large list of issues linked both to the effective action of the basic substances employed and the real presence of molecules able to balance the salivary acidity of hematophagous insects, without damaging in this way the area exposed to the substance.

After-bite devices that use only the heat action to denature the anticoagulant and vasodilating protein injected by the biting insect are also commercially available.

Even such alternative devices are not without disadvantages. In fact, it has been found that the exclusive use of heat on an area already compromised by a sting can generate, particularly in cases of skin hypersensitivity, further discomfort, and thus be disadvantageous.

### SUMMARY OF THE INVENTION

The main object of the present invention is to provide an after-insect bite device and method adapted to overcome the drawbacks above mentioned with reference to the after-bite devices known to the state of the art.

Another object of the present invention is to provide an after-insect bite device, which can be easily made and produced at reasonable costs.

Yet another object of the present invention is to provide an after-insect bite device, which can be simply and immediately used by subjects of all ages, at any time of day and throughout the year.

Yet another object of the present invention is to provide an after-insect bite device and method, which are capable of eliminating anti-aesthetic skin marks associated with continuous stimulation of the bitten area.

Not last object of the present invention is to provide an after-insect bite device, which is widely available in pharmacies, drugstores, supermarkets, tobaccos and any other authorized retailer.

Consequently, the invention relates to an after-insect bite device and method according to independent claims 1 and 16. Preferred features are reported in the dependent claims.

In particular, the invention provides, in a first aspect thereof, an after-insect bite device comprising: a container body; an applicator head extending from one end of said container body; and a reservoir housed within the container body and adapted to contain a mixture that is intended to be applied, through the applicator head, onto a part affected by the insect bite.

The device is characterized in that the mixture is a mixture of completely vegetal substances and in that the device further comprises heating means, which can be activated by switching on/off means to heat the part affected by the insect bite, before applying the mixture.

In this after-insect bite device, the issues outlined above are solved by the combined action of the mixture of completely vegetal substances and the heat generated by the heating means. In fact, the interaction between the heat and the mixture contained in the reservoir has the synergistic effect of relieving itching, due to the natural substances, namely essential oils, present in the mixture, meanwhile speeding up the curing and healing process of the injured part, as well as completely denaturing the substances that cause itching and irritation, so as to obtain, advantageously, harmless substances for the body.

Preferably, the device comprises a control unit housed within the container body and configured to heat the heating means to a denaturation temperature varying from about 60° C. to about 90° C. This advantageously allows to increase the thermal exchange with the part affected by the sting, meanwhile avoiding possible burns of the bitten subject.

In an alternative embodiment, the control unit comprises a timer configured to maintain the denaturation temperature for a predetermined time varying between 3 and 10 seconds, preferably of 5 seconds. This advantageously allows to have an effective heat exposure range of the part affected by the sting.

Preferably, the device comprises power supply means of the heating means which are housed within the container body and comprise a battery, more preferably a rechargeable battery.

Preferably, the reservoir comprises a valve for dispensing, preferably nebulizing, a predetermined amount of the mixture onto the part affected by the insect bite, after heating the part.

Preferably, the dispensing valve can be activated by the switching on/off means of the device, more preferably by dedicated activation means.

Preferably, the predetermined amount of the mixture which is dispensed onto the part affected by the insect bite is from about 0.02 to about 0.05 ml.

Preferably, the device further comprises an insulating element, which is housed within the container body between the control unit and the heating means and is configured to thermally insulate the control unit and the heating means therebetween.

In a preferred embodiment, the heating means consist of an electric resistance which is seated in a longitudinal hole formed in the applicator head.

In a further embodiment, the device comprises a closing plug of one end of the container body which is opposite to the end from which the applicator head extends.
Preferably, the mixture consists of essential oils dispersed in an inert solvent, preferably water.

Preferably, the essential oils can be selected from: Mimosa extract, Calendula extract, Althea extract, Alcanna extract, Bugleweed extract, Goji berry extract, Blackcurrant extract, Plantago Lanceolata extract, Jujube extract, Lavender essential oil, Blumea essential oil, and combinations thereof.

In an embodiment, the mixture further includes other components that can be selected from: menthyl lactate, rennet, whey, vitamin D, vitamin E, hyaluronic acid and combinations thereof.

Preferably, the mixture further comprises an antioxidant agent.

In a second aspect thereof, the invention provides an after-insect bite method comprising the following steps:

a) heating a part affected by the insect bite; and
b) dispensing a predetermined amount of a mixture of completely vegetal substances onto the heated part.

Preferably, the method comprises a step of awaiting between the heating and dispensing steps.

Preferably, the part affected by the insect bite is heated to a denaturation temperature, varying from about 60° C. to about 90° C.

Preferably, the predetermined amount of mixture dispensed onto the part affected by the insect bite is from about 0.02 to about 0.05 ml.

Preferably, the awaiting step has a duration comprised between about 3 and about 10 seconds, more preferably of 5 seconds.

Preferably, the dispensed mixture consists of essential oils dispersed in an inert solvent, preferably water.

### BRIEF DESCRIPTION OF THE DRAWINGS

Further features and advantages of the present invention will become clearer from the following detailed description of a preferred embodiment thereof, made hereinafter as indicative and non-limiting example with reference to the attached drawings. In the drawings:

FIG. 1 is a longitudinal cross-sectional view of the after-insect bite device according to the present invention;

FIG. 2 is an exploded view of the after-insect bite device of FIG. 1;

FIG. 3 is a flowchart showing the operation of the after-insect bite device of FIGS. 1 and 2;

FIGS. 4a, 4b and 4c illustrate a part affected by an insect bite before and after the use of an after-bite device using only heat according to the prior art; and

FIGS. 5a, 5b and 5c illustrate a part affected by an insect bite before and after the use of a device according to the present invention.

### DETAILED DESCRIPTION OF THE INVENTION

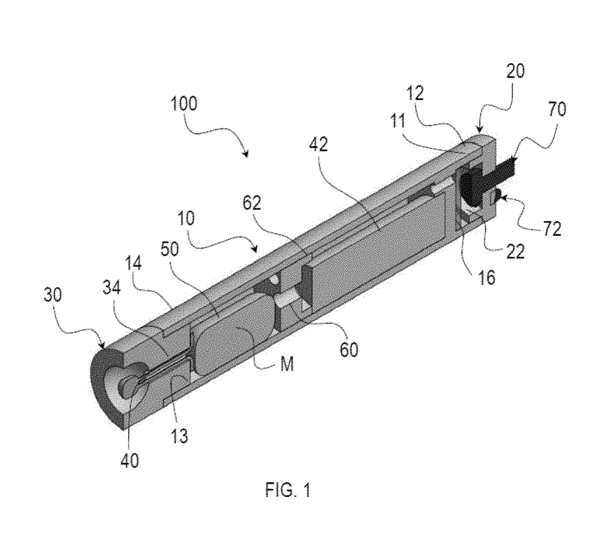

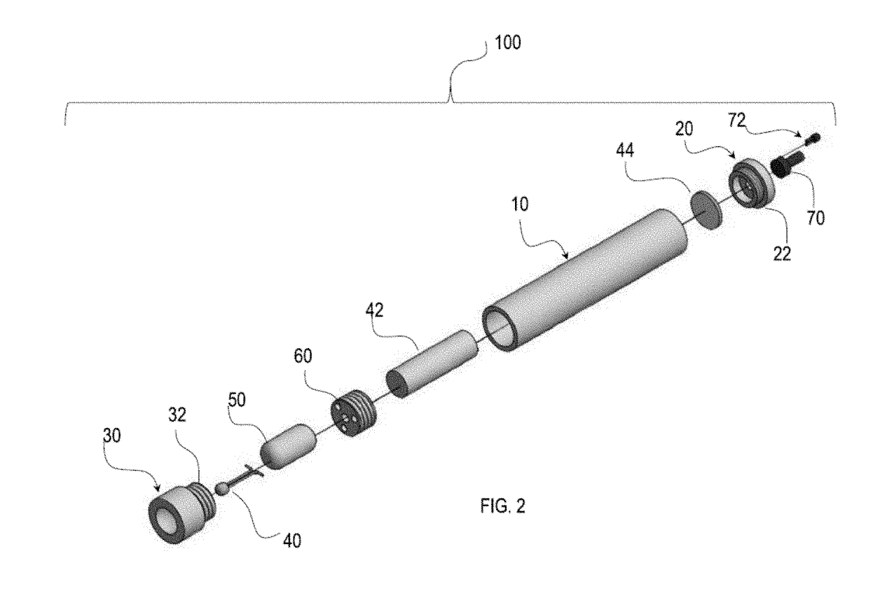

With reference to FIGS. 1 and 2, it is illustrated a preferred embodiment of an after-insect bite device according to the present invention.

The after-bite device, generally designated with the reference numeral 100, comprises a container body 10, preferably cylindrical in shape, a closing plug 20 of one end 12 of the container body 10 and an applicator head 30, extending from the other end 14 of the container body 10.

The closing plug 20 comprises a male threaded portion 22 to be screwed onto a corresponding female threaded portion 11 formed at the end 12 of the container body 10, inside the container body 10.

Similarly, the applicator head 30 comprises a male threaded portion 32 to be screwed onto a corresponding female threaded portion 13 formed at the end 14 of the container body 10, inside the container body 10.

Of course, the coupling between the container body 10 and, respectively, the closing plug 20 and the applicator head 30 can be carried out in any other known way suitable to the scope, for example by snap coupling, pressure coupling and the like.

The device 100 further comprises heating means 40 housed inside the container body 10 and which can be activated by switching on/off means of the device 100, for example a switch 70 provided externally to the container body 10. Such heating means 40 have the function to heat the part affected by the insect bite before applying onto said part a mixture M of completely vegetal substances, which will be described in greater detail in the following description.

The prior heating of the part affected by the bite increases, advantageously, the absorption capacity of the mixture M, more particularly of the essential oils contained therein, this resulting in a reduction, or a complete elimination, of the discomfort sensation generated by the bite.

Moreover, as already mentioned above, the heat produced by the heating means 40 denatures the anticoagulant and vasodilating protein present in the insect saliva and that the insect, typically a mosquito, injects under the skin.

As shown in greater detail in FIG. 1, such heating means consist of an electrical resistance 40, which is seated in a longitudinal seat 34 formed in the applicator head 30.

The electrical resistance 40 is further supplied by respective supplying means, for example a battery 42 housed inside the container body 10. The battery 42 can be of the rechargeable type.

Preferably, the temperature of the resistance 40 must be maintained within a range of from about 60° C. to about 90° C. A temperature within the above range will be indicated, in the following, as denaturation temperature T_{den}. In fact, it has been observed that the anticoagulant and vasodilating protein injected by the insect tends to denature at a temperature greater than 60° C., while in a short time a resistance temperature of 90° C. has been found to be the most suitable for increasing the thermal exchange with the part affected by the bite, meanwhile avoiding possible burns of the bitten subject.

To this end, the device 100 comprises a control unit 44, which in the embodiment shown consists of a printed circuit 44 housed inside the container body 10 between the closing plug 20 and the battery 42. More specifically, the control unit 44 is accommodated in a circular seat 16 formed in the inner surface of the container body 10.

After pressing the switch 70, the control unit 44 operates the resistance 40 until the denaturation temperature T_{den} is reached and maintains this denaturation temperature T_{den} for a predetermined time t.

To this end, the control unit 44 includes a timer, which is activated when the resistance 40 reaches the denaturation temperature T_{den} and allows this temperature T_{den} to be maintained for the predetermined time t, after which the electric resistance 40 is switched off.

Preferably, the device 100 further comprises indicator means, for example a LED (Light Emitting Diode) 72, provided externally to the container body 10 and configured to switch on when the device 100 is activated and subsequently to switch off when the time t has elapsed. Switching off the LED 72 informs the user that it is the time to dispense the mixture M on the part affected by the bite, as will be described in detail below.

An interchangeable reservoir 50 suitable for containing the mixture M of completely vegetal substances is also accommodated in the housing body 10.

Preferably, the reservoir 50 is placed within the container body 10 between the resistance 40 and the battery 42 and is in fluid communication with the applicator head 30 of the device 10 through a dispensing valve 52. The opening of the valve 52, which results in nebulization of a predetermined quantity of the mixture M onto the part affected by the bite, is controlled by the switch 70 or, in an alternative embodiment, by dedicated operating means, for example an additional switch also provided externally to the container body 10.

An insulating body 60 is preferably housed inside the container body 10 to isolate the battery 42 and control unit 44 from the resistance 40. Damaging heating or melting of plastic material and heat dispersion are thus prevented, with the heat being only directed on the part to be treated. In particular, a circular seat 62 for housing a terminal portion of the battery is formed in the insulating body 60.

The mixture M comprises essential oils dispersed in an inert solvent, typically water, extracts of natural substances, at least one stabilizer and at least one emulsifier.

Preferably, the mixture M further includes an antioxidant agent, such as ascorbic acid or vitamin C, to further stabilize the active ingredients present in the oils or vegetal extracts by means of an antioxidant process.

Essential oils, or ethereal oils, are substances of complex composition, present in the form of tiny droplets in flower petals, in fruit peels, in resin and bark of trees and roots of aromatic herbs and plants. In plants, in addition to promoting the pollination process, essential oils have a protective function against bacterial, fungi, and mold infections, meanwhile promoting healing of injured plant tissues.

These essential oils are, therefore, natural products which, once extracted, appear as oily, liquid, volatile substances and are scented as the plant from which they originate.

Since they are not water soluble, their mixture is dissolved in oil, to be then suspended, by means of a stabilizer, in water.

This advantageously allows to avoid the use of alcohol as a solvent, which is irritating and causes burning, especially in exposed areas, such as the skin after an insect bite.

The molecules of essential oils have a great affinity with human body tissues, i.e. they penetrate easily into the skin, through the skin pores, thus entering the systemic circulation through the blood, or through the lymph and interstitial fluid.

The function of essential oils suspended in the inert solvent varies from extract to extract, being a wide combination of said essential oils possible.

In the following, a list of essential oils and/or extracts that can be used in the device 100 of the present invention, together with properties and indications thereof, is provided.

Mimosa extract: healing, antiseptic, and dermo-purifying activity. Suitable for skin lesions, burns or erythema.

Calendula extract: re-epithelizing, healing, dermo-purifying, and insect repellent activity. Suitable for the treatment of burns or dermatoses.

Althea extract: emollient, antiseptic (disinfectant), and healing activity. Suitable for the treatment of infections.

Alcanna extract: re-epithelizing, healing activity. Suitable for the treatment of skin lesions.

Bugleweed extract: antiseptic and healing activity. Suitable for the treatment of skin lesions.

Goji berry extract: high content of vitamin C and also B. Antioxidant, tonic, dermo-purifying activity. Suitable for the treatment of dermatosis.

Blackcurrant extract: immune-stimulating and dermo-purifying activity. Suitable for the treatment of dermatosis, and also to relieve itching.

Plantago Lanceolata extract: hemostatic, coagulant, antiseptic and dermo-purifying activity. Suitable for the treatment of infections or dermatoses.

Jujube extract: it contains vitamin C and A. Moisturizing and dermo-purifying activity. Suitable for the treatment of dermatosis or itching.

Blumea essential oil: it contains many other essential oils having beneficial properties, such as linalool (about 19%, highly present in rose wood) and limonene. Suitable for the treatment of skin lesions.

Lavender essential oil: countless chemical analyzes on lavender essential oil have highlighted the many and varied components, each of which can act specifically on different problems or symptoms. Among these, the following are to be mentioned: linalyl acetate, linalool, lavandulyl acetate, lavandulol, and cineole which is strongly antiseptic, dermo-purifying and healing, if applied to burns, wounds, sores and insect bites. It also has a soothing action on itching, and is used to treat dermatosis.

Preferably, to increase the effectiveness of the mixture M, other components, also of completely vegetal origin, and always at low percentages, for example ranging from 0.01% to 1%, are added to it.

Such additional components include: menthyl lactate, rennet, milk whey, vitamin D, vitamin E, hyaluronic acid and combinations thereof.

Menthyl lactate is produced from two natural ingredients, namely menthol and lactic acid. It has high ability to relieve itching.

Rennet has healing and dermo-purifying properties, and it is particularly suitable for skin lesions or dermatosis. Milk whey has moisturizing, nutrient, emollient and hydrating properties, and it is particularly suitable for counteracting signs of time and skin dryness.

Vitamin D has the essential role to promote calcium fixation in the bones. It is also able to act in other districts of the human body, such as muscles, eyes, heart, lungs, or on cell proliferation, as its receptor is present everywhere in our body. Vitamin D also plays an important role, taking part to the synthesis of ceramides, in a better differentiation of the stratum corneum cells, and the maturation of the binding that holds the skin layers together: it has therefore a healing activity.

Vitamin E has soothing, moisturizing, and elasticizing properties. It is particularly effective in the treatment of acne and eczema, where it favors healing. It contains tocopherol, a strong lipid soluble antioxidant.

Hyaluronic acid is the natural component of connective tissues and, in the skin, plays an important structural role, along with collagen and elastin. It has no contra-indications and promotes healing.

### Example 1: Mixture M

In the following Table 1, a formulation example of the mixture M to be used in the device 100 according to the invention is provided.

TABLE 1

| Component | Property | Active ingredients | % |
|---|---|---|---|
| water | Inert solvent | | 42.5 |
| peg 40 idrogenated oil | solubiliser | | 20 |
| peg 6 caprylic capric | emulsifier | | 5 |
| triglycerides | surfactant | | |
| glycerin | wetting | | 5 |
| disodium | surfactant | | 5 |
| cocoamphodiacetate | | | |
| lemon-orange juice | preservative, | | 5 |
| | antioxidant | | |
| menthyl lactate | cooling | | 1 |
| officinalis calendula oil | re-epithelizing, | unspecified bitter substance, malic | 2 |
| | healing, | acid, triterpenic glycosides, | |
| | dermo-purifying, | triterpenic alcohols, sterols, | |
| | skin-protecting, | carotenoids, flavonoids and | |
| | antiseptic, | coumarins, essential oil, | |
| | insect repelling, | polysaccharides, mucilage, gums, | |
| | immune-stimulant | resins, phenols, pyrogenic tannins, | |
| | | vitamin C, manganese | |
| seabuck seed oil | healing, | oil with unsaturated fatty acids, | 2 |
| | antioxidant, | vitamin C, carotenoids, triterpenes, | |
| | antiseptic, | sterols, flavonoids, oxalic acids, | |
| | immune-stimulant, | citric, tartaric, chlorogenic, malic, | |
| | vasoprotective | succinic, tocopherol, pectin | |
| cetrimonium bromide | cationic surfactant | | 1 |
| | in case of need to | | |
| | maintain the pH | | |
| | stable | | |
| disodium edta | buffer | | 1 |
| lavender oil | antiseptic, dermo- | linalool, linalyl acetate, limonene, | 0.5 |
| | puriying, calming, | cineole, camphor, α-terpineol, β- | |
| | skin-protective, | ocimens; pernicious alcohol, | |
| | insect repelling | tannins, ursolic acid, flavonoids, | |
| | | bitter substances | |
| vitamin D | healing, | | 0.2 |
| | antimicrobial, | | |
| | melanin regulator, | | |
| | anticancer | | |
| | activity, | | |
| | it facilitates | | |
| | endorphin release | | |
| goji berry extract or juice | antioxidant, | amino acids in large quantities, | 0.1 |
| | dermo-purifying, | betaine pigments, β-carotene, | |
| | skin-protective, | vitamins B1, B2, C, nicotinic acid, | |
| | tonic, antiseptic, | scopoletin, taurine, trace minerals, | |
| | healing, immune- | cinnamic acid, fats, sugars | |
| | stimulant, re- | | |
| | epithelizing | | |
| for lavanda: linalool, | possible oil and/or | list B of cosmetic supplements: few | 0.1 |
| limonene | extract allergens | consumer reports, low risk | |

Of course, the percentages reported in Table 1 are only indicative, since the amount of the mixture components is potentially variable based on the strengthen of the purchased product.

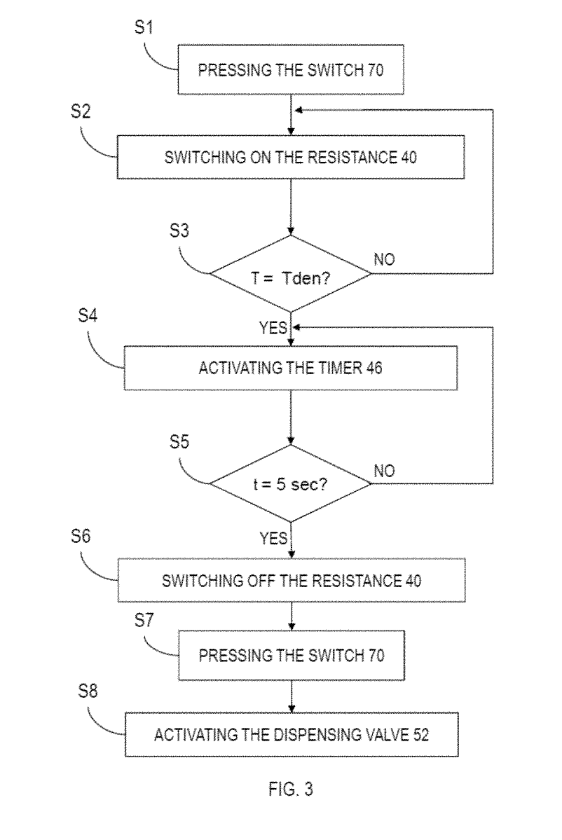

With reference to FIG. 3, it is now described the operation of the after-insect bite device 100 according to the present invention.

It is assumed that a subject has been bitten by an insect, for example a common mosquito.

As disclosed above, during the bite, the mosquito injects into the human body an anticoagulant and vasodilating protein present in its saliva. As this protein is extraneous to the body of the bitted subject, it is detected as an antigen and causes an obvious immune response, which manifests in the appearance of a wheal and an itching sensation.

Once the wheal appears, the bitten subject can use the device 100 not only to relieve the annoying itching, but also to speed up the curing and healing process of the part affected by the bite.

To this end, the bitten subject puts the applicator head 30 of the device 100 in contact with the part affected by the bite and activates the device 100 by operating the switch 70 (step S1).

Following the activation of the device 100, the control unit 44 activates the resistance 40 until the denaturation temperature T_{den} is reached (step S2).

Upon reaching the temperature T_{den} (step S3, YES), the control unit 44 activates the timer 46 (step S4) so that this temperature T_{den} is maintained for a predetermined time t of from about 3 seconds to about 10 seconds, preferably of 5 seconds.

After the time t has lapsed (step S5, YES), the control unit 44 controls the switching off of the resistance 40 (step S6), with consequent switching off of the LED 72. This advantageously allows to obtain an effective range of heat exposure of the part affected by the bite. In fact, heat applied by the device 100 causes skin pores to dilate, thus making the latter more permeable to the subsequent passage of the substances contained in the mixture M.

Once it is heated to the temperature T_{den} for the predetermined time t, the part is then ready to receive the mixture M contained in the reservoir 50 of the device 100.

At this point, the subject may press again the switch 70 or, according to a further embodiment, an additional switch also provided outside the device 100 (step S7), thereby opening the valve 52 of the reservoir 50 and subsequent nebulizing a predetermined dose, preferably 0.02-0.05 ml, of the mixture M (step S8).

According to a further embodiment, the opening of the valve 52 is automatically controllable by the control unit 44, specifically by the timer switching off, without a further pressure of the switch 70 being necessary by the bitten subject.

The mixture M, and more particularly the essential oils present therein, penetrate optimally in the heated skin, resulting in the restoration of the “normal” condition of the part affected by the bite, that is, the disappearance of the wheal. Furthermore, thanks to the presence of menthyl lactate in the mixture M, the bitten subject feels on the skin a sense of relief and freshness that eliminates any feeling of discomfort related to the mosquito bite or the use of heat.

### Example 2

In the following example, a wheal W caused by a mosquito bite was treated by using a heat emitting device according to the prior art (Comparative 1), a mixture consisting of essential oils dispersed in an inert solvent (Comparative 2), and a device 100 according to the present invention filled with a mixture M consisting of essential oils dispersed in an inert solvent (Invention).

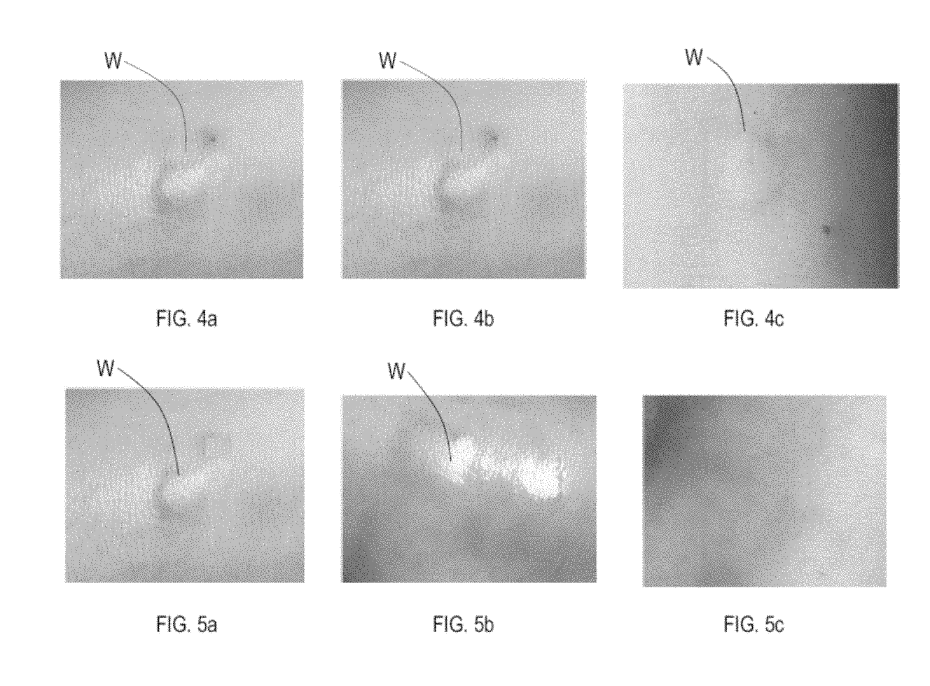

FIG. 4a shows the wheal W before the treatment with a heat emitting device and FIG. 5a shows the wheal W before the treatment with a device according to the present invention.

The wheal size was observed in two instants following the treatment, respectively after 7 minutes and 15 minutes. In the following Table 2 the results of the above observations are reported.

TABLE 2

| Treatment | 7 min | 15 min |
|---|---|---|
| Comparative 1 | The wheal size was unchanged | The wheal size was |
| | | slightly reduced |
| Comparative 2 | The wheal size was unchanged | The wheal size was |
| | | unchanged |
| Invention | The wheal disappeared | — |

By treating the wheal W with the heat emitting device, after 7 minutes the wheal size was unchanged (FIG. 4b) and after 15 minutes the wheal size was slightly reduced, but it did not disappear (FIG. 4c).

By treating the wheal W with the mixture consisting of essential oils dispersed in an inert solvent, after 7 minutes the wheal size was unchanged and after 15 minutes the wheal size was still unchanged.

Finally, by treating the wheal W with the device 100 according to the present invention (FIG. 5b) after 7 minutes the wheal W disappeared, as shown in FIG. 5c, together with the itching.

The above disclosed Example 2 clearly shows that the interaction between the heat emitted by the device 100 and the mixture of completely vegetal substances M contained in the device 100 produces the synergistic effect of speeding up the curing and healing process of the injured part, as well as completely denaturing the substances that cause itching and irritation.

From the description, they are clear the features of the after-insect bite device and the steps of after-insect bite method of the present invention, as well as the relative advantages thereof.

Further variants of the embodiments described above are possible without departing from the teachings of the invention.

1. After-insect bite device comprising:
a container body;
an applicator head extending from one end of said container body;
a reservoir housed within the container body and adapted to contain a mixture (M) that is intended to be applied, through said applicator head, onto a part affected by the insect bite;
characterized in that said mixture (M) is a mixture (M) of completely vegetal substances and in that the device further comprises heating means housed within the container body and which can be activated by switching on/off means of said device to heat said part affected by the insect bite, before applying said mixture (M).

2. Device according to claim 1, further comprising a control unit housed within said container body and configured to heat the heating means to a denaturation temperature (Tden) varying from about 60° C. to about 90° C.

3. Device according to claim 2, wherein said control unit comprises a timer configured to maintain said denaturation temperature (Tden) for a predetermined time (t) varying from 3 and 5 seconds.

4. Device according to claim 3, wherein said predetermined time (t) is of 5 seconds.

5. Device according to claim 1, further comprising power supply means of said heating means, which are housed within said container body and comprise a battery.

6. Device according to claim 1, wherein said reservoir comprises a valve for dispensing a predetermined amount of said mixture (M) onto the part affected by the insect bite, after heating said part.

7. Device according to claim 6, wherein said predetermined amount of said mixture (M) which is dispensed onto the part affected by the insect bite is from about 0.02 to about 0.05 ml.

8. Device according to claim 6, wherein said dispensing valve is activated by said switching on/off means.

9. Device according to claim 2, further comprising an insulating element, which is housed within the container body between said control unit and said heating means and is adapted to thermally insulate said control unit and said heating means therebetween.

10. Device according to claim 1, wherein said heating means consist of an electric resistance inserted in a longitudinal hole formed in said applicator head.

11. Device according to claim 1, further comprising a closing plug of one end of said container body which is opposite to the end from which said applicator head extends.

12. Device according to claim 1, wherein said mixture (M) consists of essential oils dispersed in an inert solvent.

13. Device according to claim 12, wherein said essential oils can be selected from: Mimosa extract, Calendula extract, Althea extract, Alcanna extract, Bugleweed extract, Goji berry extract, Blackcurrant extract, Plantago Lanceolata extract, Jujube extract, Lavender essential oil, Blumea essential oil, and combinations thereof.

14. Device according to claim 1, wherein said mixture (M) further includes other components that can be selected from: menthyl lactate, rennet, milk whey, vitamin D, vitamin E, hyaluronic acid, and combinations thereof.

15. Device according to claim 1, wherein said mixture (M) further comprises an antioxidant agent.

16. After-insect bite method comprising the following steps in sequence:
a) heating a part affected by the insect bite; and
b) dispensing a predetermined amount of a mixture (M) of completely vegetal substances onto said heated part.

17. Method according to claim 16, comprising a step of awaiting between said heating and dispensing steps.

18. Method according to claim 16 or 17, wherein said part affected by the insect bite is heated to a denaturation temperature (Tden), varying from about 60° C. to about 90° C.

19. Method according to claim 16, wherein said predetermined amount of said mixture (M) dispensed onto the part interested by the insect bite is from about 0.02 to about 0.05 ml.

20. Method according to claim 17, wherein said awaiting step has a duration comprised between about 3 and about 10 seconds.

21. Method according to claim 16, wherein said dispensed mixture (M) consists of essential oils dispersed in an inert solvent.
